(19) United States
(12) Patent Application Publication
(10) Pub. No.: US 2006/0177505 A1
(43) Pub. Date: Aug. 10, 2006

(54) Cleaning composition in solid form

(75) Inventor: Agnes Calduk, Fontenay-Aux-Roses (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/336,844

(22) Filed: Jan. 23, 2006

Related U.S. Application Data
(60) Provisional application No. 60/661,903, filed on Mar. 16, 2005.

(30) Foreign Application Priority Data
Jan. 31, 2005 (FR) 05 50264

(51) Int. Cl. A61K 9/46; A61K 31/60

(52) U.S. Cl. 424/466; 514/165

(57) ABSTRACT

Cleansing composition provided in solid form and containing, in an anhydrous medium, at least one active agent chosen from salicylic acid and its derivatives, at least one pulverulent foaming surfactant, and at least one water-soluble polymer, it being possible for the quantity of water-soluble polymer to be up to 10% of the total weight of the composition. The composition is preferably provided in powdered or tablet form and it may be incorporated into a sponge. The composition may be used in particular for cleansing greasy skin with a tendency towards acne.

### REFERENCE TO PRIOR APPLICATIONS

This application claims priority to U.S. provisional application 60/661,903 filed Mar. 16, 2005, and to French patent application 0550264 filed Jan. 31, 2005, both incorporated herein by reference.

### FIELD OF THE INVENTION

The invention relates to a composition, preferably one used and suited for cleansing the skin, provided in the form of a solid and in particular the form of a powder, comprising at least one active agent chosen from salicylic acid and salicylic acid derivatives (sometimes called a salicylic acid compounds herein), at least one pulverulent foaming surfactant, and at least one water-soluble polymer, and to its use for cleansing the skin of the body and of the face, including around the eyes.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing the skin is very important for facial care. It should be as efficient as possible because fatty residues such as excess sebum, the residues of cosmetic products used daily and makeup products, in particular waterproof products, accumulate in the skin folds and can block the pores of the skin and cause the appearance of spots.

It is known to incorporate into skin cleansing compositions keratolytic active agents and in particular salicylic acid or a salicylic acid derivative, these active agents making it possible to cleanse the skin in depth and to have an action on comedones in the case of skin with a tendency towards acne. Cleansing compositions are most often provided in the form of more or less liquid aqueous compositions containing foaming surfactants.

However, when such a composition contains a high percentage of foaming surfactants (for example a higher % of surfactants), it is difficult to incorporate therein a high percentage of keratolytic active agent and in particular of salicylic acid because of the problem of solubility or of pH because these active agents, although they remain stable themselves, can destabilize the aqueous composition containing them and cause a drop in viscosity, and can sometimes even cause yellowing of the composition containing them.

In addition, it is difficult to have a foaming composition containing a large quantity of surfactant and salicylic acid which is at the same time well tolerated and which is comfortable.

The need therefore remains for a composition containing a high percentage of surfactants and which may contain salicylic acid or one of its derivatives, without a problem of formulation or of safety arising.

### SUMMARY OF THE INVENTION

The inventor has found that it is possible to obtain a cleansing composition which overcomes the disadvantages of the prior art, by preparing a solid anhydrous cleansing composition containing pulverulent anionic surfactants and at least one water-soluble polymer.

### DETAILED DESCRIPTION OF THE PREFERRED EMBODIMENTS

One subject of the present invention is a solid anhydrous cleansing composition comprising at least one active agent chosen from salicylic acid and its derivatives, at least one pulverulent foaming anionic surfactant, and at least one water-soluble polymer. In a preferred embodiment it is possible for the quantity of water-soluble polymer to be up to 10% of the total weight of the composition.

The expression “composition in solid form” is understood to include, among other things commonly understood by those of ordinary skill in this art, compositions in the form of a tablet (solid having a size of up to about 4 cm) and of a powder, a powder being a solid that is finely divided into particles (it being possible for the particles to have a size which may range, for example, from 50 to 600 μm), as opposed to a composition in the form of a cream or a gel which are flexible compositions.

Moreover, the expression “pulverulent” is understood to mean a compound or a composition provided in powdered form (made up of particles or grains).

The composition according to the invention can be prepared by simply mixing the constituents which for example may all be pulverulent; it is therefore easy to manufacture. In addition, it has excellent solubility in water and it has good foaming properties, while being comfortable and well tolerated, as the test presented later shows.

Moreover, the expression “physiologically acceptable medium” is understood to mean a medium compatible with keratin materials, and in particular the skin.

The composition of the invention may constitute a cosmetic or dermatological composition.

Salicylic Acid and Derivatives

The composition of the invention comprises at least one of salicylic acid and salicylic acid compounds. Preferably, they are provided in powdered form.

As salicylic acid compounds, use may be made of any derivative which may for example be used in a cosmetic and/or dermatological composition, and in particular the salicylic acid compounds of formula (I) or a salt of such a derivative: 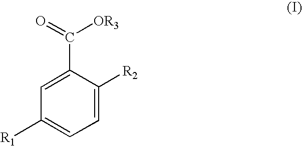
in which:
R₁ represents hydrogen or a saturated, linear, branched or cyclized aliphatic, alkoxy, ester or ketoxy chain, an unsaturated chain carrying one or more conjugated or unconjugated double bonds, these chains containing from 1 to 22 carbon atoms and being capable of being substituted with at least one substituent chosen from halogen atoms, the trifluoromethyl group, the hydroxyl groups in free form or esterified with an acid having from 1 to 6 carbon atoms and alternatively with a carboxyl functional group, which is free or esterified with a lower alcohol having from 1 to 6 carbon atoms;
R₂ represents a hydroxyl group or an ester functional group of formula (II): 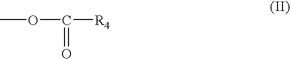
where R₄ represents a saturated aliphatic group or an alkenyl group having from 1 to 18 carbon atoms; R₃ represents hydrogen or a saturated or unsaturated linear or branched chain having from 2 to 30 carbon atoms, optionally containing one or more substituents. The chain may be chosen in particular from alkyl and alkenyl radicals containing from 2 to 30 carbon atoms, which are optionally substituted. The substituent may be in particular a hydroxyl radical.

When R₃ is hydrogen, it is also possible to use salts of the acids of formula (I), and in particular salts obtained by salification with a base.

As a base which is capable of salifying the salicylic acid derivatives of formula (I), there may be mentioned, without limitation, inorganic bases such as alkali metal hydroxides (sodium and potassium hydroxides) or ammonium hydroxides or better still the organic bases such as primary, secondary, tertiary or cyclic organic amines and more especially amino acids. By way of example of bases, there may be mentioned glycine, lysine, arginine, taurine, histidine, alanine, valine, cysteine, trihydroxymethylaminomethane (TRISTA) and triethanolamine.

According to a specific embodiment of the invention, as derivative of formula (I), those where the radical R₁ contains at least 4 carbon atoms are used in the composition of the invention are preferred. It is for example formed of a saturated linear alkyl or alkoxy radical having from 4 to 11 carbon atoms.

As derivatives of formula (I) in which R₁ is formed of a saturated linear alkyl or alkoxy radical having from 4 to 11 carbon atoms, R₂ is a hydroxyl and R₃ is hydrogen, mention may be made for example of 5-n-octanoylsalicylic (CTFA name: Capryloyl Salicylic Acid), 5-n-decanoylsalicylic, 5-n-dodecanoylsalicylic, 5-n-octylsalicylic, 5-n-heptyloxysalicylic, 5-tert-octylsalicylic, 5-butoxysalicylic, 5-ethoxysalicylic, 5-methoxysalicylic, 5-propoxysalicylic, 5-methylsalicylic, 5-ethylsalicylic and 5-propylsalicylic acids, and mixtures thereof, these acids being generally salified with a base, the compounds described in the document EP-A-570230 may also be used.

When R₁ represents hydrogen and R₂ a hydroxyl group, the derivative of formula (I) is a salicylic acid ester. It includes preferably esters of fatty alcohols such as esters of dodecyl, hexadecyl, stearyl, cetyl, myristyl, linoleyl, octyl, oleyl and tridecyl alcohols, or also esters of butyl, propyl and ethyl alcohols or alternatively the esters of polyols such as the esters of propylene glycol, butylene glycol, ethylene glycol or glycerol, or mixtures of these esters. This may include in particular cetyl salicylate, dodecyl salicylate and tridecyl salicylate.

According to a preferred embodiment of the invention, the active agent is salicylic acid.

The quantity of salicylic acid and/or its derivatives is not limited and may range for example from 0.1 to 30% by weight, preferably from 0.5 to 10% by weight and better still from 0.5 to 5% by weight relative to the total weight of the composition.

Foaming Anionic Surfactant

The composition according to the invention comprises at least one pulverulent foaming anionic surfactant, that is to say at least one foaming anionic surfactant in powdered form and/or paste form. The quantity of foaming surfactant(s) is not limited and may range for example from 5 to 99.9% by weight, preferably from 10 to 99% by weight and better still from 20 to 98% relative to the total weight of the composition.

According to a preferred embodiment of the invention, the composition comprises, as foaming anionic surfactant, at least one fatty acid salt. The fatty acid salts which constitute soaps are derived from a fatty acid having an alkyl chain containing from 6 to 22 carbon atoms, preferably from 8 to 18 carbon atoms. This includes in particular salts obtained by neutralizing a fatty acid with an organic or inorganic base such as potassium hydroxide, sodium hydroxide, triethanolamine, N-methylglucamine, lysine and arginine. As fatty acid salts (soaps), there may be mentioned in particular the alkali metal salts and for example the potassium or sodium salts of lauric, myristic, palmitic and stearic acids (potassium or sodium laurate, myristate, palmitate and stearate), in particular potassium laurate and potassium myristate, and more particularly potassium myristate which is preferably used as soap in the context of the present invention.

It is also possible to use, as pulverulent anionic surfactants, alkali metal salts of N-acylamino acids, alkyl sulphates, alkyl ether sulphates, sulphonates and mixtures thereof.

As alkali metal salts of N-acylamino acids, there may be mentioned in particular sarcosinates, alaninates, glutamates, aspartates and mixtures thereof.

The surfactants in powdered form or in paste form include those chosen for example from sodium lauroyl sarcosinate like the product marketed under the name Sarkosyl NL 97 by the company Ciba; sodium myristoyl sarcosinate such as the product marketed under the name Nikkol Sarcosinate MN by the company Nikkol, sodium palmitoyl sarcosinate such as the product marketed under the name Nikkol Sarcosinate PN by the company Nikkol, sodium lauryl sulphate such as the product marketed under the name Empicol LZ D by the company Allbright & Wilson or under the name Tensopol USP97 by the company Tensachem; sodium lauroyl glutamate such as the product marketed under the name Amisoft LS 11 by the company Ajinomoto; monosodium myristoyl glutamate such as the product marketed under the name Acylglutamate MS 11 by the company Ajinomoto; the mixture of sodium laureth sulphate and silica, marketed under the name Texapon KE 2713 by the company Cognis; disodium cocamido MEA-sulphosuccinate such as the product marketed under the name Mackanate CM 100 by the company MacIntyre; sodium methyl cocoyl taurate such as the product marketed under the name Tauranol WSP by the company Finetex; sodium decyl d-galactoside uronate such as the product marketed under the name sodium d-decyl d-galactoside uronate by the company Ard-Soliance; lauroyl methyl beta-alanine (acid form) marketed under the name LMA-H by the company Mitsui Toatsu; n-lauroyl-n-hydroxyethyl-beta-alanine marketed under the name LHEA by the company Mitsui Toatsu; sodium cocoyl glycinate marketed under the name Amilite GCS-11 (F) by the company Ajinomoto; sodium cocoyl isethionate such as the product marketed under the name Jordapon CI P by the company BASF; the sodium salt of palm kernel oil methyl taurate such as the product marketed under the name Hostapon CT PATE® by the company Clariant; sodium N-cocoyl N-methyl taurate such as the product marketed under the name Hostapon LT-SF® by the company Clariant or Nikkol CMT-30-T® by the company Nikkol; sodium palmitoyl methyl taurate such as the product marketed under the name Nikkol PMT® by the company Nikkol; sodium lauryl sulphoacetate such as the product marketed under the name Lathanol LAL powder by the company Stepan; potassium myristate such as the product marketed under the name potassium myristate (DUB MK) by the company Stearinerie Dubois; potassium laurate such as the product marketed under the name potassium laurate (DUB LK) by the company Stearinerie Dubois, and sucrose laurate such as the product marketed under the name Grilloten LSE 87 by the company Degussa.

According to a preferred embodiment of the invention, the composition contains, as foaming anionic surfactants, one or more fatty acid salts, alone or as a mixture with one or more isethionate salts such as sodium cocoyl isethionate. According to a particularly preferred embodiment of the invention, the composition contains, as foaming anionic surfactants, potassium myristate and sodium cocoyl isethionate.

The composition may additionally optionally contain one or more nonionic or amphoteric surfactants, such as for example sugar esters; alkyl polyglucosides (APG), preferably those containing an alkyl group containing from 6 to 30 carbon atoms and preferably from 8 to 16 carbon atoms, and containing a hydrophilic group (glucoside) preferably comprising 1.2 to 3 saccharide units; betaines. As nonionic or amphoteric surfactants, there may be mentioned in particular cocamidopropylbetaine such as the product marketed under the name Tegobetain CK D by the company Degussa and sucrose laurate such as the product marketed under the name Grilloten LSE 87 by the company Degussa.

Water-Soluble Polymer

The composition according to the invention comprises at least one water-soluble polymer. It may contain several thereof. In a preferred embodiment the total quantity of water-soluble polymers may be up to 10% of the total weight of the composition (i.e., a positive amount less than or equal to 10% of the total weight of the composition, or said differently, a positive amount of up to and including 10% of the total weight of the composition).

The expression “water-soluble polymer” is understood to mean a polymer which swells or dissolves in water at room temperature (temperature of about 20 to 25° C.).

This water-soluble polymer may be chosen in particular from polysaccharides among which gums, synthetic polymers, cellulose derivatives and mixtures thereof.

By way of example, useful water-soluble polymers which can be used in the invention include guar, xanthan, carrageenan, cellulose and sclerotium gums, and derivatives of these gums; hydroxyalkylcelluloses and sodium carboxycelluloses; polyacrylamides and copolymers of acrylamides; gelatine; agar; carboxyvinyl polymers such as the products marketed under the names carbopol by the company Noveon (CTFA name: carbomer), modified carboxyvinyl polymers and in particular acrylate/C₁₀-C₃₀ alkyl acrylate copolymers such as the products marketed under the name Pemulen TR1 or TR2 or CARBOPOL 1382 by the company Noveon (CTFA name: acrylates/C10-30 Alkyl acrylate Crosspolymer).

According to a preferred embodiment of the invention, the composition contains a pulverulent water-soluble polymer, more particularly a gum and in particular xanthan gum.

The quantity of water-soluble polymer(s) in the composition of the invention is not limited and for example may be up to 10% by weight relative to the total weight of the composition. This quantity may range for example from 0.01 to 10% by weight, preferably from 0.1 to 10%, better still from 0.5 to 10% by weight, and even better from 1 to 5% by weight relative to the total weight of the composition.

Additives

The composition of the invention may contain one or more additives. Such additives preferably are anhydrous or in solid form (powder), and include in particular those generally used in the cosmetic and dermatological fields, such as for example sequestrants, perfumes, antioxidants, active agents other than salicylic acid and its derivatives, preservatives, colorants (such as pigments and hydrophilic colorants) and inorganic fillers and/or organic fillers such as modified starch such as that marketed under the name Dry Flo by the company National Starch. When they are present, these additives may be in any quantity, such as a quantity ranging from 0.1 to 50%, and preferably from 0.5 to 50% of the total weight of the composition.

The composition according to the invention may also contain one or more oils or oily active agents, but preferably in a quantity of less than or equal to 5% of the total weight of the composition.

Of course, persons skilled in the art will be careful to choose this or these optional additives and/or their quantities such that the advantageous properties intrinsically attached to the composition in accordance with the invention are not, or not substantially, impaired by the addition(s) envisaged.

As active agents, there may be mentioned for example ascorbic acid (vitamin C).

According to a particular embodiment of the invention, the composition is free of polyol (glycerine, glycols) or contains a quantity thereof which is less than 5% of the total weight of the composition.

The composition of the invention can be prepared by simply mixing the constituents in a mixing tank, avoiding any contact with water or moisture.

Advantageously, the composition of the invention contains a physiologically acceptable medium, that is to say a medium compatible with keratin materials and in particular with the skin and/or the hair. It may constitute in particular a cosmetic or dermatological composition.

The composition of the invention gives rise to the formation of foam in the presence of water and exhibits in particular good efficacy for cleansing the skin.

Accordingly, the subject of the present invention is also the use of the composition according to the invention for cleansing the skin and/or the hair.

Another subject of the present invention is a method for cleansing the skin, which comprises wetting the composition with water, in passing it over the skin, and in rinsing the skin (for example as one would with a traditional bar of soap).

The composition according to the invention may also be used for the treatment of greasy skin, especially that with a tendency towards acne, which skin generally has skin imperfections, dilated pores, a nonhomogeneous skin texture and red blotches.

Thus, a subject of the present invention is also a method for the treatment of skin with a tendency towards acne, comprising the topical application of the abovementioned composition to the skin.

Moreover, the composition according to the invention may also be used for cleansing skin having acne before any treatment.

Another subject of the invention is the use of the composition as defined above for the preparation of a dermatological composition intended for the treatment of acne.

According to a particular embodiment of the invention, the composition of the invention, whether it is in powdered or tablet form, or some other solid form, is incorporated into a sponge or a similar article (e.g., swab, wipe, etc.) having a cavity or other capacity to hold the invention composition, for example as a single dose presentation. To use it, the sponge, etc. is moistened and then it is passed over the face, skin, etc., followed by rinsing.

Another subject of the present invention is a sponge containing a composition according to the invention, and its use for cleansing the skin.

Examples of compositions in accordance with the invention are given below. The quantities are given as a percentage by weight.

### Example 1

### Cleansing Composition

| | |
|---|---|
| Sodium cocoyl isethionate | 45.5% |
| Xanthan gum | 3% |
| Potassium myristate | 45.5% |
| Salicylic acid | 2% |
| Perfume | 4% |

The composition was obtained by mixing the constituents which were all in powdered form. It was provided in the form of a powder. A quantity of 0.7 g of this powder was introduced into a round cotton wool sponge having a diameter of about 70 mm.

This sponge is used by wetting it with water, passing it over the skin to be cleansed and rinsing.

The test performed on 42 women during 4 weeks of application with one application per day showed that:

for 100% of the women, the foam was abundant;

for 95% of the women, the sponge cleansed in depth;

for 97% of the women, the cleansing left the skin clean and purified;

for 69% of the women, the pores were less visible;

for 88% of the women, the imperfections were eliminated;

for 95% of the women, the composition was easy to rinse off;

for 74% of the women, the cleansing left the skin comfortable (without tightness).

### Example 2

### Cleansing Composition

| | |
|---|---|
| Sodium cocoyl isethionate | 45.5% |
| Xanthan gum | 3% |
| Aluminium starch octenyl succinate (filler) | 45.5% |
| Salicylic acid | 2% |
| Perfume | 4% |

The composition was prepared as in Example 1. A composition was obtained in powdered form. The composition was prepared as in Example 1. A composition was obtained in powdered form. A quantity of 1 g of this powder was compressed in tablet form and was introduced into a sponge which was used in a manner similar to that of Example 1.

The above written description of the invention provides a manner and process of making and using it such that any person skilled in this art is enabled to make and use the same, this enablement being provided in particular for the subject matter of the appended claims, which make up a part of the original description and including a composition provided in solid form and containing, in an anhydrous medium, at least one active agent chosen from salicylic acid and its derivatives, at least one pulverulent foaming anionic surfactant and at least one water-soluble polymer, it being possible for the quantity of water-soluble polymer to be up to 10% of the total weight of the composition.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A solid anhydrous composition comprising:
at least one active agent chosen from salicylic acid and salicylic acid compounds,
at least one pulverulent foaming anionic surfactant, and
at least one water-soluble polymer, wherein said water-soluble polymer is present in said composition in a positive amount of up to and including 10% of the total weight of the composition.

2. The composition according to claim 1, wherein the total amount of salicylic acid and its compounds ranges from 0.1 to 30% by weight relative to the total weight of the composition.

3. The composition according to claim 1, comprising at least one salicylic acid compound of formula (I) or a salt of such a compound: 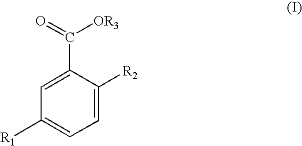
in which:
R₁ represents hydrogen or a saturated, linear, branched or cyclized aliphatic, alkoxy, ester or ketoxy chain, an unsaturated chain carrying one or more conjugated or unconjugated double bonds, these chains containing from 1 to 22 carbon atoms and optionally substituted with at least one substituent chosen from halogen atoms, a trifluoromethyl group, hydroxyl groups in free form or esterified with an acid having from 1 to 6 carbon atoms and alternatively with a carboxyl functional group, which is free or esterified with a lower alcohol having from 1 to 6 carbon atoms;
R₂ represents a hydroxyl group or an ester functional group of formula (II): 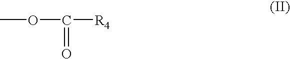
where R₄ represents a saturated aliphatic group or an alkenyl group having from 1 to 18 carbon atoms;
R₃ represents hydrogen or a saturated or unsaturated linear or branched chain having from 2 to 30 carbon atoms, optionally containing one or more substituents.

4. The composition according to claim 1, comprising at least one salicylic acid compound chosen from 5-n-octanoylsalicylic, 5-n-decanoylsalicylic, 5-n-dodecanoylsalicylic, 5-n-octylsalicylic, 5-n-heptyloxysalicylic, 5-tert-octylsalicylic, 5-butoxysalicylic, 5-ethoxysalicylic, 5-methoxysalicylic, 5-propoxysalicylic, 5-methylsalicylic, 5-ethylsalicylic and 5-propylsalicylic acid.

5. The composition according to claim 1, wherein the quantity of foaming anionic surfactant(s) is 5 to 99.9% by weight relative to the total weight of the composition.

6. The composition according to claim 1, comprising one or more alkali metal salts of a fatty acid.

7. The composition according to claim 1, comprising one or more alkali metal salts of a fatty acid and one or more isethionate salts of a fatty acid.

8. The composition according to claim 1, wherein the water-soluble polymer is chosen from polysaccharides, synthetic polymers, celluloses and mixtures thereof.

9. The composition according to claim 1, comprising xanthan gum.

10. The composition according to claim 1, wherein the quantity of polymer(s) is 0.01 to 10% of the total weight of the composition.

11. The composition according to claim 1, wherein it is provided in tablet or powdered form.

12. The composition according to claim 1, wherein the salicylic acid compounds are chosen from compounds of formula (I), mixtures thereof, salts of such compounds, and mixtures thereof: 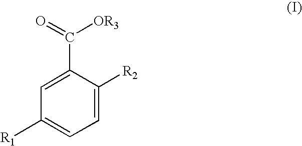
in which:
R₁ represents hydrogen or a saturated, linear, branched or cyclized aliphatic, alkoxy, ester or ketoxy chain, an unsaturated chain carrying one or more conjugated or unconjugated double bonds, these chains containing from 1 to 22 carbon atoms and optionally substituted with at least one substituent chosen from halogen atoms, a trifluoromethyl group, hydroxyl groups in free form or esterified with an acid having from 1 to 6 carbon atoms and alternatively with a carboxyl functional group, which is free or esterified with a lower alcohol having from 1 to 6 carbon atoms;
R₂ represents a hydroxyl group or an ester functional group of formula (II): 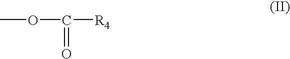
where R₄ represents a saturated aliphatic group or an alkenyl group having from 1 to 18 carbon atoms;
R₃ represents hydrogen or a saturated or unsaturated linear or branched chain having from 2 to 30 carbon atoms, optionally containing one or more substituents.

13. The composition according to claim 1, wherein the salicylic acid compounds are chosen from 5-n-octanoylsalicylic, 5-n-decanoylsalicylic, 5-n-dodecanoylsalicylic, 5-n-octylsalicylic, 5-n-heptyloxysalicylic, 5-tert-octylsalicylic, 5-butoxysalicylic, 5-ethoxysalicylic, 5-methoxysalicylic, 5-propoxysalicylic, 5-methylsalicylic, 5-ethylsalicylic 5-propylsalicylic acid and mixtures thereof.

14. A sponge comprising a composition according to claim 1.

15. A method for cleansing the skin and/or the hair, comprising applying the composition of claim 1 to the skin and/or the hair.

16. A method, comprising wetting the composition according to claim 1 with water, passing the wetted composition over the skin, and rinsing the skin.

17. The method according to claim 16, wherein said skin is skin with a tendency towards acne.

18. The method of claim 15, wherein said composition is located in and/or on a sponge.

19. The method of claim 16, wherein said composition is located in and/or on a sponge.

20. The method of claim 17, wherein said composition is located in and/or on a sponge.
